(19) United States
(12) Patent Application Publication
(10) Pub. No.: US 2015/0321983 A1
(43) Pub. Date: Nov. 12, 2015

(54) A PROCESS FOR THE PREPARATION OF OSPEMIFENE

(71) Applicant: Fermion Oy, Espoo (FI)

(72) Inventor: Jan TOIS, Espoo (FI)

(73) Assignee: Fermion Oy, Espoo (FI)

(21) Appl. No.: 14/436,690

(22) PCT Filed: Oct. 17, 2013
(86) PCT No.: PCT/FI2013/000040

§ 371 Date: Apr. 17, 2015

Related U.S. Application Data
(60) Provisional application No. 61/716,175, filed on Oct. 19, 2012.

(51) Int. Cl. C07C 41/26

(52) U.S. Cl. CPC C07C 41/26

(57) ABSTRACT

The present invention is related to the process for the preparation ospemifene or (Z)-2[4-(4-chloro-1,2-diphen-yl-but-1-enyl)phenoxy]ethanol (I) and to intermediate compounds used in the process.

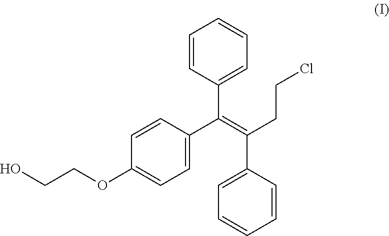 [SMILES: OCCOc1ccc(/C(=C(/CCCl)c2ccccc2)c2ccccc2)cc1]

### FIELD OF THE INVENTION

The invention is related to a process for the preparation ospemifene and to intermediate compounds used in the process.

### BACKGROUND OF THE INVENTION

Ospemifene or (Z)-2-[4-(4-chloro-1,2-diphenyl-but-1-enyl)phenoxy]ethanol is represented by formula (I):

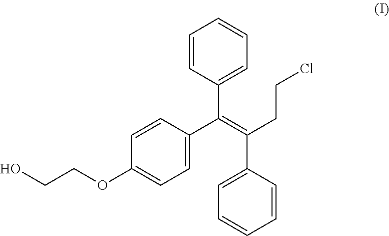 [SMILES: OCCOc1ccc(/C(=C(/CCCl)c2ccccc2)c2ccccc2)cc1]

Ospemifene is an estrogen receptor agonist/antagonist currently investigated e.g. for the treatment of vulvar and vaginal atrophy due to menopause.

Preparation of ospemifene starting from Z-4-(4-hydroxy-1,2-diphenyl-but-1-enyl)phenol has been described in WO 96/07402. Use of McMurry coupling reaction wherein two ketone groups are coupled to produce an alkene compound has been suggested for the manufacture of ospemifene. WO 2008/099059 describes McMurry coupling, between commercially available starting materials, 4-hydroxybenzophenone (or (4-hydroxyphenyl)(phenyl)methanone) and 3-chloropropiophenone, to produce mainly Z-isomer of 4-(4-chloro-1,2-diphenyl-but-1-enyl)phenol intermediate of ospemifene. McMurry coupling between 4-(2-hydroxyethoxy)benzophenone and 3-chloropropiophenone to give mainly Z-isomer of the end product (ospemifene) has been described in WO 2011/089385.

McMurry coupling reaction is known to be susceptible to side reactions as two molecules of the same starting material react with each other (homocoupling). It was found that the above described McMurry processes for preparing ospemifene suffer from the drawback that the hydroxyl substituted end product of the McMurry coupling reaction is cumbersome to isolate from the homocoupling impurities, (VIIₐ and VII_{b}),

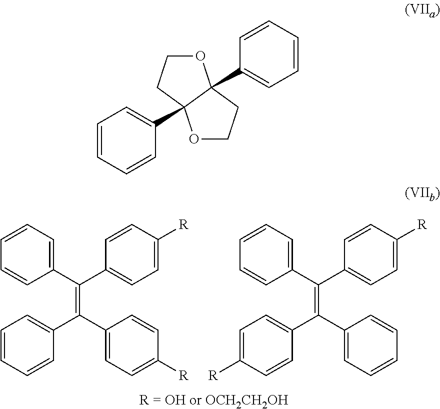

that are formed in the reaction, particularly if high yield of the end product is desired.

Thus, it is desirable to provide an improved method for producing ospemifene in high yield and purity, the method also being economically feasible, operationally practical and suitable for use in a large scale.

### SUMMARY OF THE INVENTION

The present invention provides a process for the preparation of a compound of formula (I)

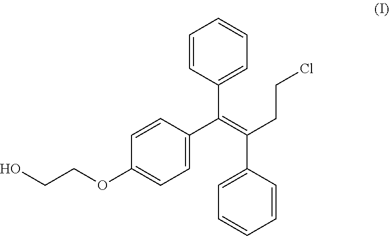 [SMILES: OCCOc1ccc(/C(=C(/CCCl)c2ccccc2)c2ccccc2)cc1]

which process comprises

(a) reacting a compound of formula (IIIₐ)

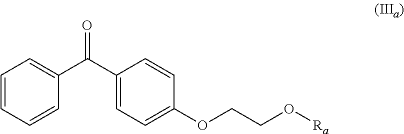

wherein Rₐ is a protective group which is benzyl, wherein the phenyl ring of the benzyl group is optionally substituted, or C(O)—R_{b}, wherein R_{b} is C₁₋₅ alkyl or an optionally substituted phenyl, with 3-chloropropiophenone to produce a compound of formula (IVₐ)

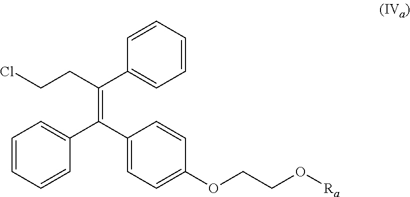

wherein Rₐ is as defined above, and

(b) subjecting the compound of formula (IVₐ) to the removal of the protective group to give a compound of formula (I)

One embodiment of the invention is a process for the preparation of a compound of formula (I)

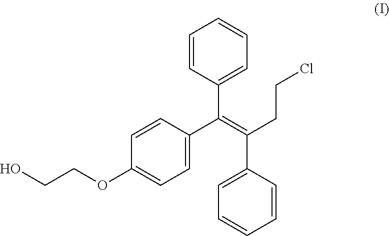 [SMILES: OCCOc1ccc(/C(=C(/CCCl)c2ccccc2)c2ccccc2)cc1]

which process comprises

a) reacting a compound of formula (III_{b})

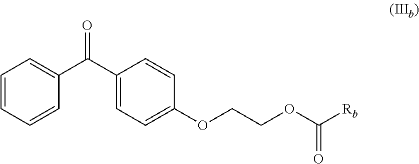

wherein R_{b} is as defined before, with 3-chloropropiophenone to produce a compound of formula (IV_{b})

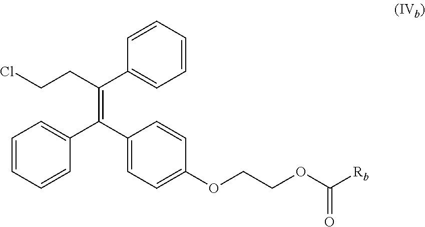

wherein R_{b} is as defined above, and

(b) subjecting the compound of formula (IV_{b}) to cleavage of the ester bond of the R_{b}—C(O)O group to give a compound of formula (I).

Another embodiment of the present invention is process for the preparation of a compound of formula (I)

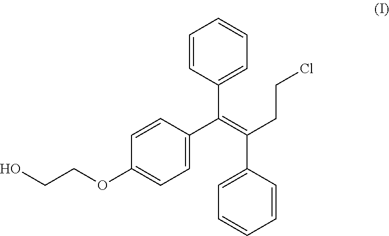 [SMILES: OCCOc1ccc(/C(=C(/CCCl)c2ccccc2)c2ccccc2)cc1]

which process comprises

(a) reacting a compound of formula (IIIₐ)

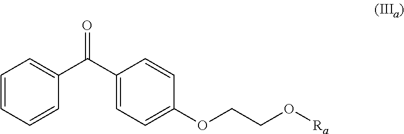

wherein Rₐ is benzyl wherein the phenyl ring of the benzyl group is optionally substituted, with 3-chloropropiophenone to produce a compound of formula (IVₐ)

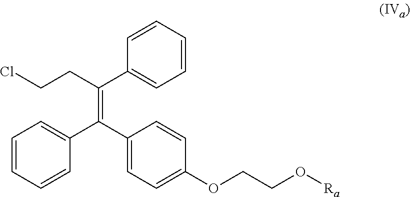

wherein Rₐ is benzyl wherein the phenyl ring of the benzyl group is optionally substituted, and

(b) subjecting the compound of formula (IVₐ) to cleavage of the ether bond to give a compound of formula (I).

In another aspect the invention provides a process for the preparation of a compound of formula (I) comprising the step of removing the protective group Rₐ from compound of formula (IVₐ).

In another aspect, the present invention provides a process for the preparation of a compound of formula (I) comprising the step of cleaving the ester bond of a compound of formula (IV_{b}), wherein R_{b} is C₁₋₅ alkyl or an optionally substituted phenyl, to give a compound of formula (I). Still in another aspect, the present invention provides a process for the preparation of compound of formula (I) comprising the step of cleaving the ether bond of a compound of formula (IVₐ), wherein Rₐ is benzyl wherein the phenyl ring of the benzyl group is optionally substituted, to give a compound of formula (I).

The invention is also directed to novel compounds of formula (IV_{b}) wherein R_{b} is C₁₋₅ alkyl or an optionally substituted phenyl, and to a novel compound of formula (III_{b}), wherein R_{b} is t-butyl.

It has been found that, in contrast, to hydroxyl substituted McMurry coupling products used earlier in the preparation of ospemifene, the compounds of formula (IVₐ) can be easily isolated in high yield and purity by a simple crystallization step. The relatively low solubility, of compounds of formula (IVₐ) to lower alcohols allows crystallization from plain lower alcohols or from a mixture of aromatic hydrocarbon and lower alcohol. Such crystallizations have, been found to effectively isolate the compound of formula (IVₐ) from its homocoupling impurities in high yield. Ospemifene can then be obtained from the compound of formula (IVₐ) by removing the protective group while the amount of homocoupling impurities, such us the impurities of formula (VIIₐ) and (VII_{b}), remains low.

### DETAILED DESCRIPTION OF THE INVENTION

The term “optionally substituted phenyl”, as used herein, refers to a phenyl ring which may be substituted by 1-3 substituents selected from C₁₋₅ alkyl and C₁₋₅ alkoxy groups. Representative examples include methyl, ethyl, t-butyl, methoxy, ethoxy and t-butoxy. Particularly preferred are methoxy and methyl substituents, especially methoxy or methyl group in 4-position, or three methyl groups in 2,4,6-positions.

The term “lower alcohol” means C₁₋₅ alcohol, preferably C₁₋₃ alcohol. Representative examples include methanol, ethanol and isopropanol. The term aromatic hydrocarbon as used herein refers to a phenyl ring which may be substituted by 1-3 substituents selected from C₁₋₅ alkyl groups. Representative example include xylenes. Particularly preferred are xylenes and toluene. Terms xylenes and xylene both refer to any of o-, m-, and p-xylene or their mixtures in all proportions.

In accordance with the present invention a compound of formula (IIIₐ)

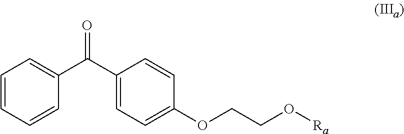

wherein Rₐ is a protective group which is benzyl, wherein the phenyl ring of the benzyl group is optionally substituted, or C(O)—R_{b}, wherein R_{b} is C₁₋₅ alkyl or an optionally substituted phenyl, is reacted with 3-chloropropiophenone to produce a compound of formula (IVₐ)

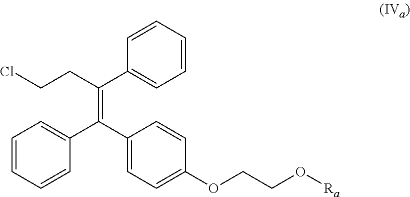

wherein Rₐ is as defined above.

The above reaction is suitably carried out in the presence of a titanium chloride, such as TiCl₃ or, preferably, TiCl₄, and a reducing agent in a suitable solvent. The suitable reducing agents include, but are not limited to, zinc powder, zinc-copper couple, potassium, magnesium and LiAlH₄. Zinc powder is a preferred reducing agent. At least one molar equivalent, more typically at least two molar equivalents, of the titanium chloride (e.g. TiCl₄) is used per compound of formula (IIIₐ). When the reducing agent is zinc, at least one molar equivalent, more typically at least two molar equivalents, of zinc is used per titanium chloride.

Suitable solvents include, but are not limited to, 2-methyltetrahydrofuran (2-Me-THF), tetrahydrofuran (THF) and dimethoxyethane (DME). Particularly preferred, solvent system is a mixture of 2-Me-THF and xylenes. The amount of solvent used is suitably between about 0.1-10 ml, more typically between about 0.5-5 ml, per mmol of compound of formula (IIIₐ).

Preferably, the reducing agent and a titanium chloride, e.g. zinc powder and TiCl₄, are added first to the reaction solvent, suitably in nitrogen atmosphere, and the mixture is refluxed for 1-2 h. The compound of formula (IIIₐ) and 3-chloropropiophenone are, then added. Preferably compound (IIIₐ) and 3-chloropropiophenone are first dissolved in xylene or a mixture of xylene and 2-Me-THF and this solution is added to the reaction. The reaction between the compound of formula (III_{c}) and 3-chloropropiophenone is preferably carried out under heating. Suitably, the reaction temperature is higher than about 50° C. preferably higher than about 60° C., for example about 70° C. or 80° C. The reaction is typically completed within less than two hours.

The compound of formula (IVₐ) is preferably isolated and purified before its use in the next reaction step. Thus, after completion of the reaction the reaction mixture is cooled, quenched with aqueous HCl solution, filtered, and the organic phase is recovered. The obtained compound of formula (IVₐ) can be easily isolated in high yield and Purity by crystallization. Thus, the organic phase is evaporated and the crystallization solvent is added. Preferably the more volatile solvent (ether) is distilled off and xylenes is left in the distillation flask and suitable crystallization solvent is added. Suitable crystallization solvents include plain lower alcohols, such as methanol, ethanol and isopropanol. Particularly suitable crystallization solvents are methanol, isopropanol and ethanol essentially in the absence of water, thereby giving the compound of formula ((IVₐ) in high yield and with low amount of homocoupling impurities such as the impurities of formula (VIIₐ) and (VII_{b}). The mixture of crystallization solvent and compound of formula (IVₐ) is stirred and suitably heated to achieve dissolution. The mixture may then be cooled to about 40° C. and seeded with the desired Z-isomer. Cooling is continued over a period of time (preferably slowly, e.g. over more than one hour) to room temperature or below, e.g. below 15° C., in order to achieve crystallization. The mixture is suitably stirred in this temperature for more than 3 hours, e.g. for 12 hours. The crystalline compound of formula (IVₐ) is filtered, washed and dried preferably under reduced pressure. The chemical purity of the crystallized compound of formula (IVₐ) is at this stage typically higher than 92% and the amount of E-isomer less than 5%. The end product may be further recrystallized if desired.

According to one embodiment of the invention, particularly suitable compounds of formula (IIIₐ) and (IVₐ) are those wherein Rₐ is C(O)—R_{b} and R_{b} is alkyl. Other particularly suitable compound are those wherein R_{b} is t-butyl. Still other particularly suitable compounds of formula (IIIₐ) and (IVₐ) are those wherein R_{b} is phenyl. According to one embodiment of the invention, particularly suitable compound of formula (IIIₐ) and (IVₐ) are those wherein Rₐ is a benzyl wherein the phenyl ring of the benzyl group is optionally substituted.

Compounds of formula (III_{b}) can be prepared using the methods known in the art

For example, compound of formula (III_{b}) can be suitably prepared by esterification of a compound of formula (II).

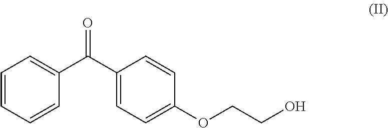 [SMILES: O=C(c1ccccc1)c1ccc(OCCO)cc1]

Esterification of a compound of formula (II) can be accomplished in numerous, ways. For example, compound of formula (II) can be reacted with an compound of formula R_{b}—C(O)-L′, wherein L′ is a suitable leaving group and wherein R_{b} is C₁₋₅ alkyl or an optionally substituted phenyl. The compound of formula R_{b}—C(O)-L′, can be in the form of a carboxylic acid, ester, acyl halide, symmetrical anhydride, mixed anhydride, phosphonium salt (as in Mitsunobu esterification) or uranium, aminium, immonium or carbonium salt. Esterification reaction can be carried out in basic, acidic or neutral conditions, and may also be carried out in the presence of coupling reagents with activation occurring in situ. Review of readily available coupling agents is provided e.g. in Valeur, E. et al., Chem. Soc. Rev., 38, 606-631, 2009. Details of various esterification reactions can be found in standard textbooks such as Greene, T. W. et al., Protective Groups in Organic Synthesis, 3. Edition, Wiley, 1999.

Specific examples of suitable leaving groups L′ for the esterification reaction include halogen and hydroxyl. In one embodiment the leaving group L′ in the compound of formula R_{b}—C(O)-L′, is halogen such as Cl. In another embodiment, the leaving group L′ is Cl and R_{b} is C₁₋₅ alkyl. In still another′ embodiment, the leaving group L′ is Cl and R_{b} is t-butyl. In still another embodiment, the leaving group L′ is Cl and R_{b} is phenyl.

According to one embodiment, the compound of formula (III_{b}) is prepared by reacting the compound of formula (II) with a compound, of formula R_{b}—C(O)—Cl, wherein R_{b} is C₁₋₅ alkyl or an optionally substituted phenyl. This esterification reaction is suitably carried out by dissolving the compound of formula (II) in suitable organic solvent such as dichloromethane (DCM) or xylenes together with a base such as triethylamine. The compound of formula R_{b}—C(O)—Cl is then added under cooling. The mixture may be further stirred e.g. at room temperature. The reaction is typically Completed within 12 hours or less. Reaction may be quenched by aqueous HCl solution. The organic phase is isolated, washed, filtered and evaporated to obtain the compound of formula (III_{b}). In one embodiment the evaporation can be omitted and compound of formula (III_{b}) can be stored and used as xylene solution.

As an alternative to esterification reaction, the compound of formula (III_{b}) may be prepared via Friedel-Crafts acylation by reacting a compound of formula (V)

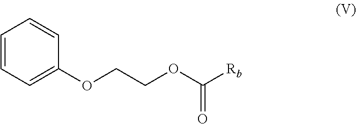

wherein R_{b} is C₁₋₅ alkyl or an optionally substituted phenyl, with a compound of formula (VI)

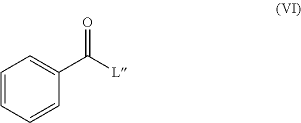

wherein L″ is a leaving group. Suitable leaving groups L″ include, but are not limited to, halogen and hydroxyl. Typically, the reaction is catalyzed by a Brønstedt acid, such as polyphosphoric acid (PPA) when L″ is hydroxyl and by Lewis acids when L″ is halogen.

According to one embodiment the compound of formula (III_{b}) is prepared by reacting the compound of formula (V), wherein R_{b} is C₁₋₅ alkyl or an optionally substituted phenyl, with benzoic acid in the presence of polyphosphoric acid (PPA). Benzoic acid and compound of formula (V), wherein R_{b} is C₁₋₅ alkyl or an optionally substituted phenyl, are suitably added to warmed PPA. Reaction mixture is stirred at elevated temperature until the reaction is complete, typically for 4 hours. Reaction is quenched with water and the mixture is extracted with suitable organic solvent such as toluene. Organic phase may then be washed, filtered and evaporated to obtain the Compound of formula (III_{b}):

Other, routes for the preparation of a compound of formula (III_{b}) are readily available for a skilled person by utilizing methods well known in the art.

Compound formula (IIIₐ); wherein Rₐ is benzyl, wherein the phenyl ring of the benzyl group is optionally substituted can be prepared using methods, known in the art. For example compound of formula (IIIₐ) can be prepared by etherification of compound of formula (II). Etherification of compound can be accomplished in numerous ways. For example compound of formula (II) can be reacted with an compound of formula RₐL′″ wherein L′″ is a suitable leaving group and Rₐ is benzyl wherein the phenyl ring of, the benzyl group is optionally substituted. The, compound of formula RₐL′″ can be in the form of alkyl halide (Cl, Br, I), alkyl sulfonate (eg. OTs, OMs, OTf) or alkyl-trichloroacetimidate (ONHCCl₃). Etherification can be carried out in basic, acidic or neutral conditions. Details of various etherification conditions can be found in standard textbooks such as Greene, T. W. et al., Protective Groups in Organic Synthesis, 3. Edition, Wiley, 1999 and Sasson, Y.; Neumann, R. Handbook of Phase Transfer Catalysis 1. Edition, Blackie Academic and Professional Chapman & Hall, 1997. In one embodiment the leaving group of the formula Rₐ-L′″ is chloride and Rₐ is benzyl. Reaction is suitable carried out in xylenes together with base and phase transfer catalyst, like described in WO 01/36360A1. After aqueous work up and concentration the compound of formula (IIIₐ) is obtained as xylene solution. Xylene solution of compound of formula (IIIₐ) can be used directly in the following step.

Ospemifene is obtained from compound of formula (IVₐ) by removing the hydroxyl protecting group Rₐ. If Rₐ is C(O)—R_{b}, ospemifene is obtained by a cleavage of the ester bond (dashed bond below)

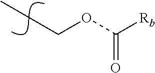

such that the hydroxyl group of ospemifene is formed.

The cleavage of the ester bond can be carried out by using well known methods such as hydrolysis or a reductive cleavage.

Hydrolysis can be catalysed by, a base or an acid. Abase catalysed hydrolysis is particularly preferred. The base catalysed hydrolysis can be carried out in a suitable solvent such as aqueous THF or aqueous THF/MeOH mixture in the presence of a suitable base, such as NaOH, KOH or LiOH at room temperature for a time sufficient to complete the hydrolysis. When the hydrolysis is carried out at room temperature, the reaction is completed typically within 12 hours or less. Thereafter, water and suitable organic solvent such as EtOAc or toluene is added. The mixture is then acidificated, the phases are separated and the organic phase is washed, dried, filtered and concentrated. Ospemifene can be conveniently isolated from the residue by crystallization from a suitable crystallization solvent. Preferred solvents for crystallization are C₁₋₅ alcohols, particularly methdriol, ethanol or isopropanol, or aqueous C₁₋₅ alcohols such as aqueous methanol (e.g. 80% or 90% methanol).

Reductive cleavage can be carried out in the presence of a reducing agent such as lithium aluminium hydride (LiAlH₄) in a suitable organic solvent such as toluene, THF, hexane or xylenes or mixture thereof. The reaction is suitably carried out at room temperature or below and under nitrogen atmosphere. The reaction may be suitably quenched by addition of saturated NH₄Cl-solution. Organic phase is washed, dried, filtered and concentrated. Ospemifene can be conveniently isolated from the residue by crystallization froth a suitable crystallization solvent as described above.

If Rₐ is benzyl wherein the phenyl ring of the benzyl group is optionally substituted, the cleavage takes place in the ether bond (dashed line below).

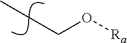

The Cleavage of the ether bond can be carried out using well known methods such as hydrogenolysis. Details of various hydrogenation conditions can be found in standard textbooks such as Greene, T. W. et al., Protective Groups in Organic Synthesis, 3. Edition, Wiley, 1999. Hydrogenolysis of the ether bond of the compound of formula (IVₐ) wherein Rₐ is benzyl wherein the phenyl ring of the benzyl group is optionally substituted, can be catalysed by transition metals. The Pd-catalyzed hydrogenolysis is particularly preferred. The catalytic hydrogenation can be carried, out in suitable solvents such as alcohols, at elevated temperatures under a pressure of hydrogen for a time sufficient to complete the hydrogenolysis. After completion of the reaction the catalyst is filtered and the filtrate is allowed to cool slowly and seeded with pure ospemifene. Cooling is continued at temperature <10° C. for more than 3 hours and crystalline ospemifene is isolated by filtration.

Ospemifene can be re-crystallized if needed from C₁₋₅ alcohols or aqueous C₁₋₅ alcohols. Ospemifene obtained by the method of the invention, has particularly high, over 99.5%, purity as it is devoid of homocoupling impurities such as the impurities of formula (VIIₐ) and (VII_{b}) typically involved in McMurry coupling reaction.

The invention is further illustrated by the following non-limiting examples.

### EXAMPLES

### Example 1

### Preparation of 2-(4-Benzoylphenoxy)ethyl pivalate

(4-(2-Hydroxyethoxy)phenyl)(phenyl)methanone (15 g, 61.9 mmol) was dissolved in dichloromethane (200 ml) and cooled on ice-bath to, 0-5° C. Triethylamine (17.26 ml, 124 mmol) was added followed by pivaloyl chloride (8.39 ml, 68.1 mmol) maintaining the temperature below 5° C. After the additions the cooling bath was removed and the mixture was stirred at room temperature (23° C.) for 12 hours. Reaction was quenched by addition of 5% HCl-solution (150 ml) and the phases were separated. Organic phase was washed with 1% NaOH-solution (2×50 ml), water (100 ml) and brine (100 ml). After drying with Na₂SO₄ the solution was filtered through a small pad of silica and concentrated in vacuo. The title compound was obtained as a yellow solid (20.1 g, 61.6 mmol, 99%). ¹H-NMR (400 MHz, CDCl₃) δ (ppm): 7.83 (2H, d, J=8.8 Hz, ArH), 7.76 (2H, m, ArH), 7.57 (1H, tt, J=7.2 Hz, J=1.2 Hz, ArH), 7.47 (2H, t, J=7.6 Hz, ArH), 6.98 (2H, d, J=9.2 Hz, ArH), 4.45 (2H, t, J=4.8 Hz, CH₂CH₂OPiv), 4.26 (2H, t, J=5.2 Hz, ArOCH₂CH₂), 1.21 (9H, s, 3×Me). ¹³C-NMR (100 MHz, CDCl₃) δ (ppm): 195.8, 178.8, 162.6, 138.6, 133.0, 132.4, 130.9, 130.1, 128.6, 114.6, 66.5, 62.8, 39.2, 27.5.

### Example 2

### Preparation of 2-(4-Benzoylphenoxy)ethyl pivalate xylene solution

(4-(2-Hydroxyethoxyl)phenyl)(phenyl)methanone (40 g, 165 moml) was suspended in xylenes (230 ml, 5.75 vol). The mixture was stirred at room temperature for 10 minutes before addition of triethylamine (33.4 g, 46 ml, 330 mmol, 2 equiv). Pivaloyl chloride (23.89 g, 24.40 ml, 198 mmol, 1.2 equiv) was added to the mixture during 30 minutes. The mixture was stirred overnight at room temperature. HPLC and TLC indicated full conversion and the mixture was filtered to remove the formed salt. The clear xylene solution was washed with 5% HCl-solution (150 ml), saturated NaHCO₃-solution (150 ml) and water (150 ml). The xylene solution was filtered through a short pad of celite. The xylene solution was transferred to distillation flask and the solution was concentrated in vacuo. The, content of 2-(4-Benzoylphenoxy)ethyl pivalate in xylene was 0.79 M and this solution was directly used in McMurry reaction described in example 5.

### Example 3

### Preparation of 2-(4-Benzoylphenoxy)ethyl pivalate

Polyphosphoric acid (25 g) was charged to a three-necked round-bottomed flask and warmed to 80° C. (bath temperature) with mechanical stirring. Benzoic acid (2.75 g, 22.52 mmol) was added to the reaction followed by 2-phenoxyethyl pivalate (6.01 g, 27 mmol). Reaction, mixture was stirred at 80° C. for 4 hours. Reaction was quenched by water (100 ml), stirred, for 2 hours and extracted, with toluene (3×25 ml). Combined toluene phases, were washed with 5% NaOH-solution (2×25 ml), water, (25 ml) and saturated NaCl-solution (2×25 ml). After filtration and concentration in vacuo crude title compound was obtained as yellow oil (6.0 g). Crude compound was triturated with hexane (50 ml) and precipitation was filtered. The title compound was obtained as white solid (2.54 g, 35%).

### Example 4

### Preparation of (Z)-2-(4-(4-Chloro-1,2-diphenylbut-1-en-1-yl)-phenoxy)ethyl pivalate and crystallization from ethanol

Zink powder (8.02 g, 123 mmol) was added to dry 2-methyltetrahydrofuran (2-Me-THF) solution (100 ml) under nitrogen atmosphere. The mixture was cooled to 0° C. and TiCl₄ (6.72 ml, 61.3 mmol) was added to the cooled mixture maintaining the temperature below 20° C. After the addition the reaction mixture was heated to 70° C. and kept at this temperature for 60 minutes. 2-(4-Benzoylphenoxy)ethyl pivalate (10 g, 30.6 mmol) and 3-chloropropiophenone (5.17 g, 30.6 mmol) were dissolved in 2-Me-THF (40 ml) and added together into the warm reaction mixture and heated further for 90 minutes at 70° C. According to HPLC full conversion was achieved and the reaction′mixture was allowed to cool at room temperature 23° C.). Water (50 ml) and 10% HCl-solution (100 ml) were added to the flask and mixture was stirred for 30 minutes. The mixture was filtered (Bühner-funnel, filterpaper) in suction and the phases were separated in a separation funnel. Aqueous phase was extracted with toluene (30 ml) and combined to 2-Me-THF-phase. Combined organic phases were washed with water (2×75 ml) and concentrated in vacuo. The crude product was crystallized from EtOH yielding the title compound as white powder (7.0 g, 49%). Chemical purity was over 92% and isomeric purity over 95%. ¹H-NMR (400 MHz, CDCl₃) δ (ppm): 7.39-7.13 (6H, m, ArH), 6.79 (2H, d, J=8.8 Hz, ArH), 6.56 (2H, d, J=8.8 Hz, ArH), 4.31 (2H, t, J=4.4 Hz, CH₂CH₂OPiv), 4.04 (2H, t, J=4.8 Hz, ArOCH₂CH₂), 3.41 (2H, t, J=7.6 Hz, ClCH₂CH₂), 2.92 (2H, t, J=7.6 Hz, ClCH₂CH₂), 1.17 (9H, s, 3×Me). ¹³C-NMR (100 MHz, CDCl₃)⁶ (ppm): 178.5, 156.8, 142.8, 141.6, 140.9, 135.3, 135.2, 131.7, 129.5, 129.4, 128.4, 128.2, 127.0, 126.6, 113.6, 65.7, 62.7, 42.8, 38.7, 38.6, 27.1.

### Example 5

### Preparation of (Z)-2-(4-(4-Chloro-1,2-diphenylbut-1-en-1-yl)-phenoxy)ethyl pivalate and crystallization from a mixture of xylenes and isopropanol

Zink powder (39.3 g, 600 mmol) was added to dry, 2-methyltetrahydrofuran (2-Me-THF) solution (500 ml) under nitrogen, atmosphere. The mixture was cooled to 0° C. and TiCl₄ (32.9 ml, 300 mmol) was added to the cooled mixture maintaining the temperature below 20° C. After the addition the reaction mixture was heated to 70° C. and kept at this temperature for 90 minutes. 3-Chloropropiophenone (25.3 g, 150 mmol) was dissolved in 2-Me-THF (70 ml) and added to the 0.79M xylene solution of 2-(4-Benzoylphenoxy)ethyl pivalate (190 ml, 49.0 g, 150 mmol) obtained in example 2. The solution was added into the warm reaction mixture and heated further for 60 minutes at 70° C. According to HPLC full conversion was achieved and the reaction mixture was allowed to cool at room temperature (23° C.). Water (300 ml) and 10% HCl-solution (300 ml) were added to the flask and mixture was stirred for 12 hours at room temperature. The mixture was filtered (Bühner-funnel, filterpaper) in suction and the phases were separated in a separation funnel. The organic phase was washed with water (2×250 ml) and filtered through a pad of celite. The clear solution was transferred to a distillation flask and the solution was concentrated in vacuo (210 mbar) to a final volume of 200 ml. Isopropanol (450 ml) was added to the xylene solution and the solution was heated to 60° C. in order to get a clear solution. The solution was cooled down slowly and seeded with pure (Z)-2-(4-(4-Chloro-1,2-diphenylbut-1-en-1-yl)phenoxy)ethyl pivalate. The mixture was left for stirring for 16 hours at room temperature and three hours at −3° C. The precipitation was filtered and washed with isopropanol (50 ml). After drying (overnight in vacuum oven at 40° C.) the weight of (Z)-2-(4-(4-Chloro-1,2-diphenylbut-1-en-1-yl)phenoxy)ethyl pivalate was 35.54 g, 51% yield. The chemical purity was 94.4a-% and isomeric purity 97.3%.

### Example 6

### Preparation of (Z)-2-[4-(4-chloro-1,2-diphenyl-but-1-enyl)-phenoxy]ethanol (ospemifene) by base, hydrolysis of the pivaloyl group

A four-necked round bottomed, flask was charged with (Z)-2-(4-(4-Chloro-1,2-diphenylbut-1-en-1-yl)phenoxy)ethyl pivalate (25 g, 54 mmol) and THF (200 ml) was added to the vessel followed by MeOH (25 ml) and Water (25 ml). To the clear solution was added KOH (3.33 g, 59.4 mmol, 1.1 equiv) in four portions. During addition the temperature of the reaction solution rose from 20° C. to 23° C. The opaque mixture was left for stirring at room temperature overnight. After 19 hours the reaction solution was totally clear, and HPLC indicated full conversion. Toluene (70 ml) was added to the reaction followed by water (100 ml). The mixture was acidified (pH 3-4) with 30% HCl-solution. The clear phases were separated and aqueous phase was re-extracted with toluene (100 ml). The organics were combined and washed with water (2×100 ml). Solution was filtered through a pad of celite and the filtrate was evaporated in vacuo. The mass of solid evaporation residue was 22 g. This crude material was re-crystallized from i-PrOH (130 ml) and the weight of obtained material was 13.7 g. After second re-crystallization from i-PrOH (90 ml) 11.7 g, yield 57% of ospemifene was obtained in more than 99.5% purity.

¹H-NMR (400 MHz, CDCl₃) δ (ppm): 7.37 (2H, t, J=8 Hz, ArH), 7.29 (3H, t, J=7.2 Hz, ArH), 7.20 (2H, t, J=7.6 Hz, ArH), 7.16-7.13 (3H, m, ArH), 6.80 (2H, J=8.8 Hz, ArH), 6.57 (2H, d, J=8.8 Hz, ArH), 3.94 (2H, t, J=4.4 Hz, ArOCH₂CH₂OH), 3.87 (2H, m, ArOCH₂CH₂OH), 3.42 (2H, t, J=7.2 Hz, ClCH₂CH₂), 2.92 (2H, t, J=7.21 Hz, ClCH₂CH₂), 1.95 (1H, t, J=6.4 Hz, OH). ¹³C-NMR (100 MHz, CDCl₃) δ (ppm): 157.2, 143.2, 142.1, 141.3, 2×135.7, 132.2, 130.0, 129.8, 128.8, 128.7, 127.4, 127.0, 113.9, 69.3, 61.8, 43.3, 39.0.

### Example 7

### Preparation of (Z)-2-[4-(4-chloro-1,2-diphenyl-but-1-enyl phenoxy]ethanol (ospemifene) by reductive cleavage of pivaloyl-group

(Z)-2-(4-(4-Chloro-1,2-diphenylbut-1-en-1-yl)phenoxy)ethyl pivalate (3.5 g, mmol) was dissolved in toluene (35 ml) and stirred under nitrogen for 5 minutes at room temperature (23° C.). Lithium aluminium hydride solution (1 M in THF) (7.56 ml, 7.56 mmol) was added dropwise to the reaction and the mixture was stirred at room temperature, for 30 minutes. After HPLC indicated completion, the reaction was quenched by addition of saturated NH₄Cl-solution (75 ml). Additional amount of toluene (30 ml) was added and the phases were separated. The organic phase was washed with water (50 ml), brine (50 ml), dried (Na₂SO₄), filtered and concentrated in vacuo. The residue was crystallized from 90% MeOH yielding ospemifene (1.75 g, 61%) as a white solid.

### Example 8

### Preparation of 2-(4-benzoylphenoxy)ethyl benzoate

A mixture of (4-(2-hydroxyethoxy) phenyl (phenyl) methanone (15 g, 61.9 mmol), benzoic acid (8.32 g, 68.1 mmol) and p-TsOH (1.18 g, 6.19 mmol) were stirred under reflux in toluene (100 ml) with simultaneous water removal for 7 h. After cooling at room temperature the reaction was quenched by addition of saturated NaHCO₃-solution (50 ml). The phases were separated and toluene phase was washed with saturated NaHCO₃-solution (50 ml), water (50 ml) and saturated NaCl-solution (50 ml). After drying (Na₂SO₄) and filtration the solution was concentrated in vacuo. The oily residue was dissolved DCM and filtered through a small pad of silica and concentrated. The title compound was obtained as a white solid (11.58 g, 33.4 mmol, 54%). ¹H-NMR (400 MHz, CDCl₃) δ (ppm): 8.06 (2H, dd, J=7.2 Hz, J=1.2 Hz, ArH), 7.84 (2H, d, J=8.8 Hz, ArH), 7.75 (2H, dd, J=8.4 Hz, J=1.6 Hz, ArH), 7.51 (2H, td, J=8.0 Hz, J=1.6 Hz, ArH), 7.49-7.43 (4H, m, ArH), 1.02 d, J=8.8 Hz, ArH), 4.71 (2H, t, J=5.2 Hz, CH₂CH₂OBz), 4.40 (2H, t, J=4.8 Hz, ArOCH₂CH₂). ¹³C-NMR (100 MHz, CDCl₃) δ (ppm): 195.6, 166.6, 162.2, 138.3, 133.3, 132.7, 132.1, 130.7, 129.8, 128.5, 128.3, 114.3, 66.3, 63.2.

### Example 9

### Preparation of 2-(4-benzoylphenoxy)ethyl benzoate

(4-(2-Hydroxyethoxy) phenyl (phenyl) methanone (5 g, 20.64 mmol) was dissolved in DCM (50 ml) and the solution was cooled on ice-bath to 0-5° C. Triethylamine (5.75 ml, 41.3 mmol) was added followed by benzoyl chloride (2.87 ml, 24.77 mmol) maintaining the temperature below 5° C. After the additions the cooling bath was removed and the mixture was stirred at room temperature for 2 h. Reaction was quenched by addition of 5% HCl-solution (25 ml) and the phases were separated. Organic phase was washed with water (25 ml), saturated NaHCO₃-solution (2×25 ml), water (25 ml) and brine (25 ml). After drying (Na₂SO₄) and filtration the solution was concentrated in vacuo. The solid residue was re-crystallized from MeOH yielding the title compound as a white solid (5.52 g, 15.94 mmol, 77%).

### Example 10

### Preparation of (Z)-2-(4-(4-chloro-1,2-diphenylbut-1-en-1-1)phenoxy)ethyl benzoate

Zink powder (3.78 g, 57.7 mmol), was added to dry 2-methyltetrahydrofuran (2-Me-THF) solution (50 ml) under nitrogen atmosphere. The mixture was cooled to 0° C. and TiCl₄ (3.17 mL, 28.9 mmol) was added to the cooled mixture maintaining the temperature below 20° C. After the addition reaction mixture was heated to 70° C. and kept at, this temperature for 90 min. 2-(4-Benzoylphenoxy)ethyl benzoate (5 g, 14.44 mmol) and 3-chloropropiophenone (2.43 g, 14.44 mmol) were dissolved in 2-Me-THF (20 ml) and added together into warm reaction mixture and heated further for 2 h at 70° C. According to HPLC full conversion was achieved and reaction mixture was allowed to cool at room temperature. Water (30 ml) and 10% HCl-solution (30 ml) were added to the flask and mixture was stirred for 30 min. The mixture was filtered (Bühner-funnel, filterpaper) in suction and the phases were separated in separation funnel. Aqueous phase was extracted with toluene (30 ml) and combined to 2-Me-THF-phase. Combined organics were washed with water (2×75 ml) and concentrated in vacuo yielding a yellowish oil. The residue was re-dissolved in a mixture of hexane and EtOAc, filtered through a small pad of silica and concentrated. According to ¹H-NMR spectrum the isomeric ratio was 4:1 (Z:E). The colourless crude product was crystallized from EtOH yielding the title compound as white powder (1.1 g, 2.28 mmol, 16%). The isomeric purity of was over 92% and chemical purity over 99.9% Second re-crystallization from EtOH increased the isomeric purity to 96.4%. ¹H-NMR (400 MHz, CDCl₃) δ (ppm): 8.03 (2H, dd, J=8.0 Hz, J=1.2 Hz, ArH), 7.55 (1H, t, J=7.2 Hz, ArH), 7.44-7.35 (4H, m, ArH), 7.31-7.26 (3H, m, ArH), 7.22-7.13 (5H, m, ArH), 6.80 (2H, d, J=8.8 Hz, ArH), 6.60 (2H, d, J=8.8 Hz, ArH), 4.58 (21-1, t, J=4.8 Hz, CH₂CH₂OBz), 4.17 (2H, t, J=4.8 Hz, ArOCH₂CH₂), 3.42 (2H, t, J=7.6 Hz, ClCH₂CH₂), 2.92 (2H, t, J=7.2 Hz, ClCH₂CH₂). ¹³C-NMR (100 MHz, CDCl₃) δ (ppm): 166.6, 156.9, 142.9, 141.8, 141.0, 135.5, 135.4, 133.2, 131.9, 130.0, 129.8, 129.7, 129.5, 128.5×2, 128.4, 127.1, 126.8, 113.7, 65.9, 63.4, 43.0, 38.7.

### Example 11

### Preparation of (Z)-2-[4-(4-chloro-1,2-diphenyl-but-1-enyl)-phenoxy]ethanol (ospemifene) by reductive cleavage of the benzoyl group

(Z)-2-(4-(4-Chloro-1,2-diphenylbut-1-en-1-yl)phenoxy)ethyl benzoate (0.7 g, 1.45 mmol) was suspended in toluene (10 ml) and stirred under nitrogen for 5 min at room temperature. Lithium aluminium, hydride solution (1 M in THF) (1.45 ml, 1.45 mmol) was added dropwise to the reaction and the mixture was at room temperature for 30 min. HPLC indicated completion and the reaction was quenched by addition of saturated NH₄Cl-solution (25 ml). Additional amount of toluene (15 ml) was added and the phases were separated. The organic phase was washed with water (25 ml), brine (25 ml), dried (Na₂SO₄), filtered and concentrated in vacuo. The residue was crystallized from 90% MeoH yielding ospemifene (0.25 g, 0.66 mmol, 45%) as a white solid. HPLC purity of crystallized Ospemifene was over 99.7 a-%.

### Example 12

### Preparation of (4-(2-(benzyloxy)ethoxy)phenyl)-(phenyl)ethanone

Prepared according to literature procedure (WO 01/36360A1) utilizing xylene as solvent. The content of (4-(benzyloxy)ethoxy)phenyl)(phenyl)methanone in xylene was 38w-%. This solution was used as obtained in McMurry reaction of example 13. For analytical sample a small volume of xylene solution was evaporated to dryness and crystallized from isopropanol. ¹H-NMR (400 MHz, CDCl₃) δ (ppm): 7.81 (2H, d, J=8.8 Hz, ArH), 7.74 (2H, m, ArH), 7.55 (1H, tt, J=8.4 Hz, J=1.2 Hz, ArH), 7.45 (2H, t, J=8.0 Hz, ArH), 7.38-7.26 (5H, m, ArH), 6.98 (2H, d, J=8.8 Hz, ArH), 4.64 (2H, s, CH₂—CH₂—O—CH₂—Ar), 4.22 (2H, t, J=4 Hz, ArO—CH₂—CH₂), 3.86 (2H, t, J=4 Hz, ArO—CH₂—CH₂). ¹³C-NMR (100 MHz, CDCl₃) δ (ppm): 195.4, 162.5, 138.3, 137.9, 132.5, 131.9, 130.3, 129.7, 128.4, 128.2, 127.8, 127.8, 114.2, 73.4, 68.3, 67.7.

### Example 13

### Preparation of (Z)-(1-(4-(2-(benzyloxy)ethoxy)phenyl)-4-chlorobut-1-ene-1,2-diyl)dibenzene and crystallization from a mixture of xylenes and isopropanol

A four-necked round bottomed flask was charged with 2-Me-THF (500 ml) and zinc powder (39.4 g, 602 mmol, 4 equiv) and stirred under nitrogen for 10 minutes. The mixture was cooled on ice-bath to −3° C. TiCl₄ (33.0 ml, 57.1 g, 301 mmol) was transferred under nitrogen atmosphere to a dropping funnel via cannula and added to the zinc-mixture during 30 minutes. The temperature rose to 16.5° C. during the addition. When addition was complete the mixture was heated at 70° C. for 90 minutes. 3-Chloropropiophenone (25.4 g, 150 mmol) was dissolved in 2-Me-THF (70 ml) and mixed with 38.2 w-% (4-(benzyloxy)ethoxy)phenyl)-(phenyl)methanone xylene solution (130.89 g solution, 50 g, 150 mmol of (4-(benzyloxy)ethoxy)phenyl)(phenyl)methanone) obtained in example 12. This solution was transferred to a dropping funnel and added during 5 minutes to the reaction. The reaction was kept at 70° C. for 60 minutes. HPLC and TLC samples were taken and both starting materials were, consumed. The heating apparatus was removed and reaction was allowed to cool at room temperature. Water (300 ml) was added and mixture was stirred for 10 minutes. 10% HCl-solution (300 ml) was added and the mixture was left for stirring overnight. The mixture was filtered through a pad of celite and transferred to separation funnel. The phases were separated and organic phase was washed with water (2×250 ml) and transferred to a four-necked round bottomed flask. The solution was concentrated in vacuo (210 mbar) and the mixture was warmed to 80° C. When the final volume was approximately 175 ml the mixture was allowed to cool at room temperature. Isopropanol (400 ml) was added and the cloudy mixture was heated to 60° C. in order to get a clear solution. The solution was cooled down slowly and seeded. The mixture was left for stirring overnight. In the following morning the mixture was cooled on ice-bath and stirred at −3° C. for three hours. The precipitation was filtered and washed with cold i-PrOH (50 ml). The precipitation was dried in suction for two hours and transferred to vacuum oven (30° C.) and dried overnight. The crude product was re-crystallized from 350 ml i-PrOH/toluene 4:1. After filtration, washing (70 ml i-PrOH) and drying 38.8 g, 51% yield of (Z)-(1-(4-(2-(benzyloxy)ethoxy)phenyl)-4-chlorobut-1-ene-1,2-diyl)dibenzene was obtained with. The chemical purity of obtained product was 94.9% and isomeric purity was over 98%. ¹H-NMR (400 MHz, CDCl₃) δ (ppm): 7.38-7.24 (10H, m, ArH), 7.19-7.10 (5H, m, ArH), 6.78 (2H, d, J=8.8 Hz, ArH), 6.57 (2H, d, J=8.8 Hz, ArH), 4.56 (2H, s, ArCH₂OCH₂), 3.98 (2H, t, J=4.4 Hz, ArOCH₂), 3.71 (2H, t, J=4.4 Hz, ArOCH₂CH₂), 3.40 (2H, t, J=7.4 Hz, ClCH₂CH₂), 2.91 (2H, t, J=7.4 Hz, ClCH₂CH₂). ¹³C-NMR (100 MHz, CDCl₃) δ (ppm): 157.0, 142.9, 141.8, 141.0, 138.0, 135.2, 135.0, 131.6, 129.5, 129.4, 128.4, 128.3, 128.2, 127.7, 127.6, 126.9, 126.6, 113.6, 73.3, 68.4, 67.2, 42.8, 38.6.

### Example 14

### Preparation of (Z)-2-[4-(4-chloro-1,2-diphenyl-but-1-enyl)-phenoxy]ethanol (ospemifene) by catalytic hydrogenation

(Z)-(1-(4-(2-(benzyloxy)ethoxy)phenyl)-4-chlorobut-1-ene-1,2-diyl)dibenzene was subjected to hydrogenation conditions according to literature procedure. After completion of the reaction (2 hours), the catalyst was filtered, the isopropanol filtrate was cooled and crystalline (Z)-2-[4-(4-chloro-1,2-diphenyl-but-1-enyl)-phenoxy]ethanol (ospemifene) was collected by filtration. After re-crystallization from isopropanol ospemifene was, obtained as white crystalline solid in 51% yield. The purity of obtained product was over 99.5%.

1. A process of preparing a compound of formula (I) 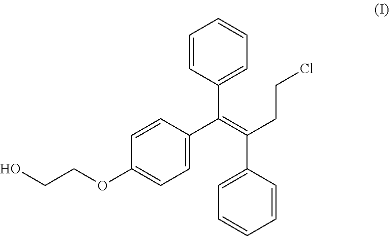 [SMILES: OCCOc1ccc(/C(=C(/CCCl)c2ccccc2)c2ccccc2)cc1]
which process comprises:
(a) reacting a compound of formula (IIIₐ) 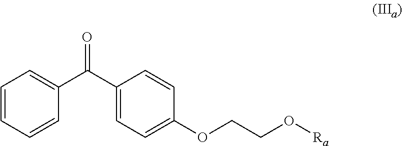
wherein Rₐ is
a protective group which is a benzyl group, wherein the phenyl ring of the benzyl group is optionally substituted, or
C(O)—R_{b}, wherein R_{b} is C₁₋₅ alkyl or an optionally substituted phenyl,
with 3-chloropropiophenome, thereby obtaining a compound of formula (IVₐ) 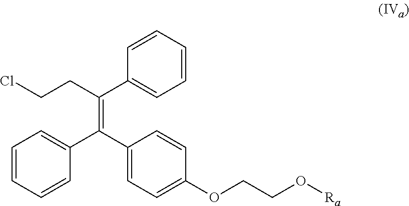
wherein Rₐ is as defined in formula (IIIₐ), and
(b) removing the protective group of the compound of formula (IVₐ), thereby obtaining a compound of formula (I).

2. The process according to claim 1, wherein Rₐ is C(O)R_{b} and the compound of formula (IIIₐ) is a compound of formula (III_{b}):

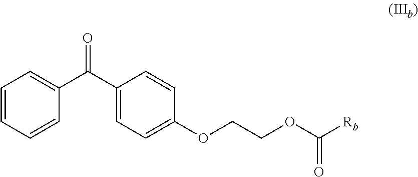

3. The process according to claim 1 wherein Rₐ is a benzyl group, wherein the phenyl ring of the benzyl group is optionally substituted.

4. The process according to claim 1, wherein (a) reacting the compound of formula (IIIₐ) with 3-chloropropiophenone is carried out in the presence of a titanium chloride and a reducing agent.

5. The process according to claim 4, wherein the reducing agent is zinc powder.

6. The process according to claim 1, wherein (a) reacting the compound of formula (IIIₐ) with 3-chloropropiophenone is carried out in 2-methyltetrahydrofuran (2-Me-THF), tetrahydrofuran (THF), or a mixture of aromatic hydrocarbon and 2-methyltetrahydrofuran as a solvent.

7. The process according to claim 1, wherein reacting the compound of formula (IIIₐ) with 3-chloropropiophenome comprises dissolving the compound of formula (IIIₐ) and 3-chloropropiophenone in xylenes or a mixture of xylenes and 2-methyltetrahydrofuran, then adding dissolved compound of formula (IIIₐ) and dissolved 3-chloropropiophenome to obtain a reaction mixture.

8. The process according to claim 1, further comprising isolating the compound of formula (IVₐ) by crystallization.

9. The process according to claim 8, wherein the isolating comprises crystallizing the compound of formula (IVₐ) from a crystallization solvent consisting of (a) C₁₋₃ alcohol or (b) a mixture of lower alcohol and aromatic hydrocarbon.

10. The process according to claim 9, wherein the isolating comprises crystallizing the compound of formula (IVₐ) from C₁₋₃ alcohol.

11. The process according to claim 10, wherein the C₁₋₃ alcohol is methanol or ethanol.

12. The process according to claim 9, wherein the crystallization solvent is a mixture of xylene and methanol or a mixture of xylene and isopropanol.

13. The process according to claim 2 wherein removing the protective group comprises cleavage of the ester bond of the R_{b}—C(O)O group in (b) by a base catalyzed hydrolysis or a reductive cleavage.

14. The process according to claim 3, wherein removing the protective group comprises cleavage of the ether bond by hydrogenolysis.

15. The process according to claim 13, wherein the cleavage of the ester bond comprises reductive cleavage in the presence of lithium aluminum hydride (LiAlH₄).

16. The process according to claim 1, further comprising isolating the compound of formula (I) by crystallization.

17. The process according to claim 16, wherein isolating the compound of formula (I) comprises crystallizing said compound of formula (I) from (a) C₁₋₅ alcohol or from (b) a mixture of C₁₋₅ alcohol and water.

18. The process according to claim 2, wherein R_{b} is C₁₋₅ alkyl.

19. The process according to claim 18, wherein R_{b} is t-butyl.

20. The process according to claim 2, wherein R_{b} is phenyl.

21. The process according to claim 2, further comprising preparing the compound of formula (III_{b})
by reacting a compound of formula (II) 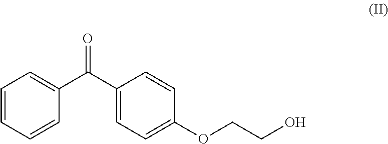 [SMILES: O=C(c1ccccc1)c1ccc(OCCO)cc1]
with a compound of formula R_{b}—C(O)-L′,
wherein L′ is a leaving group and R_{b} is C₁₋₅ alkyl or an optionally substituted phenyl.

22. The process according to claim 3, further comprising preparing the compound of formula (IIIₐ)
by reacting a compound of formula (II) 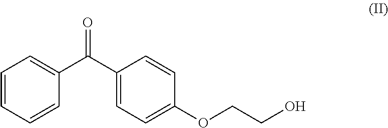 [SMILES: O=C(c1ccccc1)c1ccc(OCCO)cc1]
with a compound of formula Rₐ-L′″,
wherein L′″ is a leaving group and Rₐ is benzyl wherein the phenyl ring of the benzyl group is optionally substituted.

23. The process according to claim 2,
further comprising preparing the compound of formula (III_{b}) by reacting a compound of formula (V) 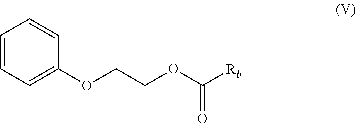
wherein R_{b} is C₁₋₅ alkyl or an optionally substituted phenyl, with a compound of formula (VI) 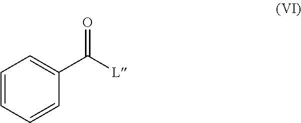
wherein L″ is a leaving group.

24. A process of preparing a compound of formula (I) 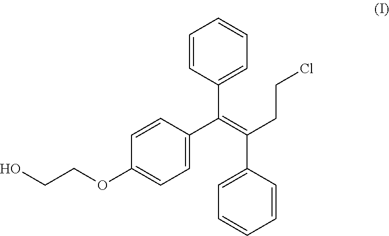 [SMILES: OCCOc1ccc(/C(=C(/CCCl)c2ccccc2)c2ccccc2)cc1]
the process comprising removing a protective group Rₐ of a compound of formula (IVₐ) 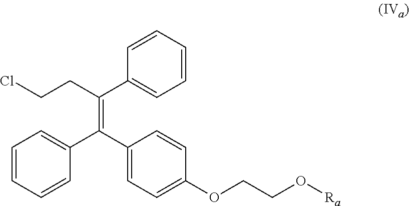
wherein the protective group Rₐ is a benzyl group comprising an optionally-substituted phenyl ring, or wherein the protective group is C(O)—R_{b}, wherein R_{b} is C₁₋₅ alkyl or an optionally-substituted phenyl.

25. A compound of formula (IV_{b}) 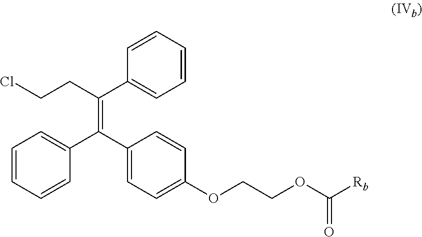
wherein R_{b} is C₁₋₅ alkyl or an optionally substituted phenyl.

26. The compound according to claim 25, wherein R_{b} is C₁₋₅ alkyl.

27. The compound according to claim 26, wherein R_{b} is t-butyl.

28. The compound according to claim 25, wherein R_{b} is phenyl.

29. A compound of formula (III_{b}) 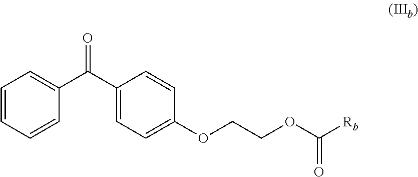
wherein R_{b} is t-butyl or phenyl.

30. The process according to claim 8, wherein a chemical purity of the compound of formula (IVₐ) immediately after the crystallization is higher than 92%.

31. The process according to claim 8, wherein the compound of formula (IVₐ) immediately after the crystallization has a content of less than 5% of an E-isomer of the compound of formula (IVₐ).

32. The process according to claim 16, wherein the compound of formula (I) has a purity of at least 99.5% immediately after the crystallization.
